(19) United States
(12) Patent Application Publication
(10) Pub. No.: US 2026/0226333 A1
(43) Pub. Date: Aug. 6, 2026

(54) BONDING RESIN

(71) Applicant: Stora Enso OYJ, Helsinki (FI)

(72) Inventors: Ashar Zafar, Älta (SE); Dimitri Areskogh, Bromma (SE); Huynh Tram Anh Pham, Älvsjö (SE)

(73) Assignee: Stora Enso OYJ, Helsinki (FI)

(21) Appl. No.: 19/138,257

(22) PCT Filed: Dec. 18, 2023
(86) PCT No.: PCT/IB2023/062860

§ 371 Date: Jun. 12, 2025

(30) Foreign Application Priority Data
Dec. 19, 2022 (SE) 2230423-2

(51) Int. Cl. C09J 197/00; C08H 7/00; C08J 3/05; C08K 5/544

(52) U.S. Cl. CPC C09J 197/005; C08H 6/00; C08J 3/05; C08K 5/544; C08J 2397/00

(57) ABSTRACT

The present invention relates to a bonding resin useful for example in the manufacture of insulation, such as mineral wool insulation or glass wool insulation. The invention also relates to a method for preparing the bonding resin and to the use thereof.

### FIELD OF THE INVENTION

The present invention relates to a bonding resin useful for example in the manufacture of insulation, such as mineral wool insulation or glass wool insulation. The invention also relates to a method for preparing the bonding resin and to the use thereof.

### BACKGROUND

Bonding resins are useful in fabricating articles, because they are capable of consolidating non- or loosely-assembled matter. For example, bonding resins enable two or more surfaces to become united. In particular, bonding resin may be used to produce products comprising consolidated fibers. Thermosetting bonding resins may be characterized by being transformed into insoluble and infusible materials by means of either heat or catalytic action. Examples of thermosetting bonding resins include a variety of phenol-aldehyde, urea-aldehyde, melamine-aldehyde, and other condensation-polymerization materials like furane and polyurethane resins. Bonding resins containing phenol-aldehyde, resorcinol-aldehyde, phenol/aldehyde/urea, phenol/melamine/aldehyde, and the like are used for the bonding of fibers, textiles, plastics, rubbers, and many other materials.

The mineral wool and fiber board industries have historically used phenol-formaldehyde bonding resins to bind fibers. Phenol-formaldehyde type bonding resins provide suitable properties to the final products; however, environmental considerations have motivated the development of alternative binders. One such alternative bonding resin is a carbohydrate-based binder derived from reacting a carbohydrate and a multiprotic acid, for example according to US2007/0027283 and WO2009/019235. Another alternative bonding resin is the esterification products of a polycarboxylic acid reacted with a polyol, for example according to US2005/0202224. Because these binders do not utilize formaldehyde as a reagent, they have been collectively referred to as formaldehyde-free binders.

One area of development is to find a replacement for the phenol-formaldehyde type binders across the entire range of products in which they are used (e.g. fiberglass insulation, particle boards, office panels, and acoustical sound insulation). In particular, the previously developed formaldehyde-free bonding resins may not possess all the desired properties for all the products. For example, acrylic acid and poly(vinylalcohol) based binders have shown promising performance characteristics. However, these are relatively more expensive than phenol formaldehyde binders, are derived essentially from petroleum-based resources, and have a tendency to exhibit lower reaction rates compared to the phenol formaldehyde based bonding resins (requiring either prolonged cure times or increased cure temperatures). Carbohydrate-based bonding resins are made of relatively inexpensive precursors and are derived mainly from renewable resources; however, these bonding resins may also require reaction conditions for curing that are substantially different from those conditions under which the traditional phenol-formaldehyde binder system cured. Therefore, replacement of phenol-formaldehyde type binders with an existing alternative has not been readily achievable.

Lignin, an aromatic polymer is a major constituent in e.g. wood, being the most abundant carbon source on Earth second only to cellulose. In recent years, with development and commercialization of technologies to extract lignin in a highly purified, solid and particularized form from the pulp-making process, it has attracted significant attention as a possible renewable substitute to primarily aromatic chemical precursors currently sourced from the petrochemical industry.

Lignin, being a polyaromatic network has been extensively investigated as a suitable substitute for phenol during production of phenol-formaldehyde adhesives. These are used during manufacturing of laminate and structural wood products such as plywood, oriented strand board and fiberboard. During synthesis of such adhesives, phenol, which may be partially replaced by lignin, is reacted with formaldehyde in the presence of either basic or acidic catalyst to form a highly cross-linked aromatic resins termed novolacs (when utilizing acidic catalysts) or resoles (when utilizing basic catalysts). Currently, only limited amounts of the phenol can be replaced by lignin due to the lower reactivity of lignin.

Tannins are phenolic-based natural products. They are found mostly in the bark of pine, the wattle of mimosa and hemlock and in the wood of certain trees such as quebracho and sumach. The extraction of these substances leads to a mixture of oligo- and poly flavonoids which are known as condensed tannins, with number average molecular weights ranging from 1000 to 4000 depending on the species which generated them. Given the phenolic-type structures borne by these oligomers, the use of tannin as macromonomers in formulations involving the characteristic phenol-formaldehyde condensation reactions has been suggested. Much experience has been gained on the making and properties of tannin-based resins. These include a number of combinations, such as phenol-formaldehyde, resorcinol-formaldehyde, urea-formaldehyde prepolymers and also mixtures therefrom to which tannins or tannin-formaldehyde resols are added: in a basic medium these mixtures cure at room temperature to networks which possess good adhesive properties, particularly for plywood. Although cold curing ensures most of the network formation, it was shown that further condensation occurs when one heats these materials at about 130° C.

A problem when preparing insulation products is to obtain appropriate strength properties, which largely depend on the bonding resin used, without the use of formaldehyde. In addition, the bonding resin should preferably be bio-based.

### SUMMARY OF THE INVENTION

It has now surprisingly been found that it is possible to easily prepare a bonding resin, suitable for use in the production of insulation, in which the use of formaldehyde can be avoided. It has also been found that the bonding resin provides improved strength properties, in particular increased modulus, making it particularly useful in the manufacture of insulation.

Further, it has been found that when lignin and/or tannin is provided in the form of an aqueous solution comprising ammonia and/or organic base, the phenolic hydroxyl groups in the lignin and/or tannin structure are deprotonated and free to react with other components of a bonding resin.

Furthermore, by providing lignin and/or tannin in the form an aqueous solution of lignin and/or tannin comprising ammonia and/or an organic base the risk of degrading for example glass wool and mineral wool fibers is minimized.

The present invention is thus directed to a bonding resin comprising a reaction product of hydroxymethylfurfural, furfural, furfuryl alcohol, acetoxymethyl furfural or an oligomer of hydroxymethylfurfural or a combination thereof, lignin and/or tannin and a polyamine, wherein the polyamine is a primary polyamine selected from a group consisting of a diamine, triamine, tetraamine and pentaamine, and wherein the polyamine is H₂N-Q-NH₂, wherein Q is C₁-C₁₀ alkyl, cycloalkyl, C₁-C₁₀ heteroalkyl, or cycloheteroalkyl, each of which is optionally substituted; and wherein the lignin and/or tannin is provided as a solution and wherein the total amount of lignin and/or tannin in the bonding resin, calculated on the basis of dry lignin and/or tannin and dry bonding resin, is in the range of from 5 wt-% to 80 wt-%.

The present invention is also directed to a fibrous insulation product comprising the bonding resin according to the present invention.

### DETAILED DESCRIPTION

It is intended throughout the present description that the expression “lignin” embraces any kind of lignin, e.g. lignin originated from hardwood, softwood or annular plants. Preferably the lignin is an alkaline lignin generated in e.g. the Kraft process. Preferably, the lignin has been purified or isolated before being used in the process according to the present invention. The lignin may be isolated from black liquor and optionally be further purified before being used in the process according to the present invention. The purification is typically such that the purity of the lignin is at least 90%, preferably at least 95%. Thus, the lignin used according to the method of the present invention preferably contains less than 10%, preferably less than 5% impurities. The lignin may then be separated from the black liquor by using the process disclosed in WO2006031175. The lignin may then be separated from the black liquor by using the process referred to as the LignoBoost process. The lignin may be provided in the form of particles, such as particles having an average particle size of from 50 micrometers to 500 micrometers.

The tannin used in the present invention is a general term of complicated aromatic compounds having a large number of phenolic hydroxyl groups. Tannins are widely distributed in the plant kingdom, and to roughly divide, tannin is divided into two kinds of a hydrolyzed type and a condensed type. Both kinds are natural compounds, and have different structures. Either tannin may be used in the present invention. Polyhydric phenol compounds having a dye-fixing effect and a tanning effect of leather are called “synthetic tannin” and “cintan”, and among the synthetic tannins, the compounds which are effectively used can be used as well in the present invention.

The reactivity of the lignin can be increased by modifying the lignin by glyoxylation, etherification, esterification, amination or any other method where lignin hydroxyl content or carboxylic content or amine content or thiol content is increased. Preferably, the lignin used according to the present invention is not modified chemically after its extraction from wood and isolation.

An aqueous solution of lignin and/or tannin comprising ammonia and/or an organic base can be prepared by methods known in the art, such as by mixing lignin and/or tannin and ammonia and/or organic base with water. The pH of the aqueous solution of lignin and/or tannin comprising ammonia and/or an organic base is preferably in the range of from 8 to 14, more preferably in the range of from 9 to 11 or 10 to 11. Examples of organic bases include amines, such as primary, secondary and tertiary amines and mixtures thereof. Preferably, the organic base is selected from the group consisting of methylamine, ethylamine, propylamine, butylamine, ethylenediamine, methanolamine, ethanolamine, aniline, cyclohexylamine, benzylamine, dimethylamine, diethylamine, dipropylamine, dibutylamine, dimethanolamine, diethanolamine, diphenylamine, phenylmethylamine, phenylethylamine, hexamethylenediamine, polyetheramine, dicyclohexylamine, piperazine, imidazole, 2-methylimidazole, 2-ethylimidazole, 2-ethyl-4-methylimidazole, 2-isopropylimidazole, 2-phenylimidazole, 2-methylimidazoline, 2-phenylimidazoline, trimethylamine, triethylamine, dimethylhexylamine, N-methylpiperazine, dimethylbenzylamine, aminomethyl propanol, tris(dimethylaminomethyl)phenol and dimethylaniline or mixtures thereof. The total amount of ammonia and/or organic base in the aqueous solution is preferably in the range of from 0.1 wt-% to 20 wt-%, preferably 0.1 wt-% to 10 wt-%, of the total weight of the aqueous solution comprising water, lignin and/or tannin and ammonia and/or an organic base. The total amount of lignin and/or tannin in the aqueous solution of lignin and/or tannin comprising ammonia and/or an organic base is preferably from 1 wt-% to 60 wt-% of the solution, such as from 10 wt-% to 30 wt-% of the solution. The aqueous solution of lignin and/or tannin comprising ammonia and/or an organic base comprises less than 1 wt-% alkali and less than 1 wt-% inorganic base. More preferably, the aqueous solution of lignin and/or tannin comprising ammonia and/or an organic base does not comprise alkali and does not comprise inorganic base.

The total amount of lignin and/or tannin in the bonding resin is preferably from wt-% to 80 wt-%, more preferably from 5 wt-% to 50 wt-% or from 5 wt-% to 30 wt-% calculated as the dry weight of lignin and/or tannin and the total weight of the bonding resin.

The hydroxymethylfurfural (HMF), furfural (FU), furfuryl alcohol (FA) or acetoxymethyl furfural is preferably provided in liquid form, preferably as an aqueous solution. In one embodiment, the aqueous solution comprising hydroxymethylfurfural (HMF), furfural (FU), furfuryl alcohol (FA) or acetoxymethyl furfural also comprises base, preferably ammonia and/or an organic base. Preferably, hydroxymethylfurfural is used according to the present invention. HMF oligomers are compounds having at least two linked HMF units/monomers. HMF oligomers preferably have a molar mass up to 3000 g/mol. HMF oligomers can be prepared according to methods known in the art, for example through a polycondensation.

An aqueous solution of hydroxymethylfurfural (HMF), furfural (FU), furfuryl alcohol (FA), acetoxymethyl furfural or an oligomer of HMF can be prepared using methods known in the art, such as by mixing hydroxymethylfurfural (HMF), furfural (FU), furfuryl alcohol (FA) or acetoxymethyl furfural and water to obtain a solution. The pH of the aqueous solution of hydroxymethylfurfural (HMF), furfural (FU), furfuryl alcohol (FA) or acetoxymethyl furfural may be in the range of from 6 to 8 or may optionally be adjusted so that the pH is in the range of from 8 to 14, more preferably in the range of from 10 to 14. Such pH adjustment is preferably carried out by addition of base. The base may be alkali or an inorganic base or preferably ammonia and/or organic base.

The amount of hydroxymethylfurfural (HMF), furfural (FU), furfuryl alcohol (FA), acetoxymethyl furfural or oligomer of HMF or combination thereof in the aqueous solution is preferably from 1 wt-% to 70 wt-% of the solution, such as from 10 wt-% to 50 wt-% of the solution, based on the dry weight of hydroxymethylfurfural (HMF), furfural (FU), furfuryl alcohol (FA), acetoxymethyl furfural or an oligomer of HMF or combination thereof and the total weight of the solution.

Thus, in a bonding resin according to the present invention the hydroxymethylfurfural (HMF), furfural (FU), furfuryl alcohol (FA), acetoxymethyl furfural or oligomer of HMF is dissolved.

The amount of hydroxymethylfurfural (HMF), furfural (FU), furfuryl alcohol (FA), acetoxymethyl furfural or oligomer of HMF in the bonding resin is preferably 10-80 wt-%, preferably 20-50 wt-%, calculated as the total dry weight of lignin and/or tannin and the total weight of the bonding resin.

As used herein, a polyamine is an organic compound having two or more amine groups. As used herein, a primary polyamine is an organic compound having two or more primary amine groups (—NH₂). Within the scope of the term primary polyamine are those compounds which can be modified in situ or isomerize to generate a compound having two or more primary amine groups (—NH₂). The polyamine is a primary polyamine.

The polyamine used in the bonding resin according to the present invention may be a molecule having the formula of H₂N-Q-NH₂, wherein Q is an alkyl, cycloalkyl, heteroalkyl, or cycloheteroalkyl, each of which may be optionally substituted. In one embodiment, Q is an alkyl selected from a group consisting of C₂-C₂₄ alkyl. In one embodiment, Q is an alkyl selected from a group consisting of C₂-C₈ alkyl. In one embodiment, Q is an alkyl selected from a group consisting of C₃-C₇ alkyl. In one embodiment, Q is a C₆ alkyl. In one embodiment, Q is selected from the group consisting of a cyclohexyl, cyclopentyl or cyclobutyl. In one embodiment, Q is a benzyl.

As used herein, the term “alkyl” includes a chain of carbon atoms, which is optionally branched. It is to be further understood that alkyl is advantageously of limited length, including C₁-C₂₄, C₁-C₁₂, C₁-C₈, C₁-C₆, and C₁-C₄. It is appreciated herein that shorter alkyl, alkenyl, and/or alkynyl groups may add less hydrophilicity to the compound and accordingly will have different reactivity and solubility.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyls include, but are not limited to, cyclopropyl, cyclopentyl, cyclohexyl, 2-methylcyclopropyl, cyclopentyleth-2-yl, adamantyl, and the like. It is to be further understood that chain forming cycloalkyl is advantageously of limited length, including C₃-C₂₄, C₃-C₁₂, C₃-C₈, C₃-C₆, and C₅-C₆. It is appreciated herein that shorter alkyl chains forming cycloalkyl may add less lipophilicity to the compound and accordingly will have different behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. In one embodiment, a heteroalkyl is a polyether. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, and/or sulfonic acid is optionally substituted.

In one embodiment of the present invention, the polyamine is selected from a group consisting of a diamine, triamine, tetraamine, and pentamine. In one embodiment, the polyamine is a diamine selected from a group consisting of 1,6-diaminohexane, 1,5-diamino-2-methylpentane and 3-(Aminomethyl)-3,5,5-trimethylcyclohexan-1-amine. In one embodiment, the diamine is 1,6-diaminohexane. In one embodiment, the polyamine is a triamine selected from a group consisting of diethylenetriamine, 1-piperazineethaneamine, and bis(hexamethylene)triamine. In one embodiment, the polyamine is a tetramine such as triethylenetetramine. In one embodiment, the polyamine is a pentamine, such as tetraethylenepentamine. In one embodiment, the primary polyamine is a polyether-polyamine i.e. an amine terminated polyether or diamines and triamines attached to a polyether backbone. In one embodiment, the polyether-polyamine is a diamine or a triamine. Examples of polyether-polyamine include polyoxypropylene triamine, polyoxypropylene diamine, triethylene glycol diamine. In one embodiment, the polyamine is selected from 1,6-diaminohexane, 1,5-diamino-2-methylpentane, hexamethylenediamine, polyetheramine, 3-(Aminomethyl)-3,5,5-trimethylcyclohexan-1-amine, diethylenetriamine, 1-piperazineethaneamine, bis(hexamethylene)triamine, triethylenetetramine and tetraethylenepentamine

The weight ratio of the lignin and/or tannin to polyamine, calculated on the basis of dry lignin and/or tannin and dry polyamine, is between 100:1 and 1:100, preferably in the range of from 20:1 to 1:20 and more preferably in the range of from 10:1 to 1:10, calculated on the basis of dry solids.

The solid content of the bonding resin before curing is preferably in the range of from 5 to 70%, such as in the range of from 15 to 50%.

The bonding resin may also comprise additives, such as urea, tannin, surfactants, dispersing agents and fillers. The bonding resin may also comprise plasticizer. In one embodiment, the bonding resin does not comprise plasticizer. As used herein, the term “plasticizer” refers to an agent that, when added to lignin and/or tannin, makes the lignin and/or tannin softer and more flexible, to increase its plasticity by lowering the glass transition temperature (Tg) and improve its flow behavior. Examples of plasticizers include polyols, alkyl citrates, organic carbonates, phthalates, adipates, sebacates, maleates, benzoates, trimellitates and organophosphates. Polyols include for example polyethylene glycols, polypropylene glycols, glycerol, diglycerol, polyglycerol, butanediol, sorbitol and polyvinyl alcohol. Alkyl citrates include for example triethyl citrate, tributyl citrate, acetyl triethyl citrate and trimethyl citrate.

Organic carbonates include for example ethylene carbonate, propylene carbonate, glycerol carbonate and vinyl carbonate. Further examples of plasticizers include polyethylene glycol ethers, polyethers, hydrogenated sugars, triacetin and solvents used as coalescing agents like alcohol ethers. In one embodiment of the present invention, the plasticizer is a polyol, such as a polyol selected from the group consisting of polyethylene glycols and polypropylene glycols. If the resin comprises a plasticizer, the weight ratio between plasticizer and lignin and/or tannin, calculated on the basis of dry weight of each component, is preferably from 0.1:10 to 10:1. Preferably, the weight ratio between plasticizer (if present) and lignin and/or tannin, calculated on the basis of dry weight of each component, is from 0.1:10 to 10:10, such as from 1:10 to 5:10. The bonding resin may also comprise coupling agent. Coupling agents are for example silane-based coupling agents. In one embodiment, the bonding resin does not comprise coupling agent.

A filler and/or hardener can also be added to the bonding resin. Examples of such fillers and/or hardeners include limestone, cellulose, sodium carbonate, and starch. In one embodiment, the bonding resin does not comprise filler and/or hardener.

Preferably, the bonding resin according to the present invention does not contain formaldehyde. Preferably, the bonding resin does not contain phenol.

Preferably, the bonding resin according to the present invention contains less than 1 wt % alkali catalyst. More preferably, the bonding does not contain alkali. Further, it is preferred that less than 1 wt-% alkali catalyst is used in the production of the bonding resin according to the present invention. More preferably, no alkali is used in the production of the bonding resin according to the present invention.

In one embodiment of the present invention, epoxy-based cross-linker is not used in the bonding resin.

The fibrous material used according to the present invention is for example mineral fibers (glass fibers, slag wool fibers, and rock wool fibers), aramid fibers, ceramic fibers, metal fibers, carbon fibers, polyimide fibers, certain polyester fibers, and rayon fibers. Such fibers are substantially unaffected by exposure to temperatures above about 120° C. In one embodiment, the insulating fibers are glass fibers. In one embodiment, the mineral fibers are present in an insulation product according to the present invention in the range from about 70% to about 99% by weight.

In one embodiment, fibrous material comprises cellulosic fibers. For example, the cellulosic fibers may be wood fibers, wood shavings, sawdust, wood pulp, or ground wood. In one embodiment, the cellulosic fibers may be other natural fibers such as jute, flax, hemp, and straw.

As used herein, the term binder solution is the solution of chemicals which can be substantially dehydrated to form an uncured bonding resin. As used herein, the bonding resin may be cured, uncured, or partially cured. The composition of the uncured bonding resin is referred to as an uncured bonding resin. An uncured bonding resin is a substantially dehydrated mixture of chemicals which can be cured to form a cured bonding resin. Substantially dehydrated means that the solvent (typically water or a mixture thereof) used to make the binder solution is vaporized to the extent that the viscosity of the remaining material (comprising the binder reactants and solvent) is sufficiently high to create cohesion between the loosely assembled matter; thus, the remaining material is an uncured bonding resin. In one embodiment, the solvent is less than 65% of the total weight of the remaining material. In one embodiment, a substantially dehydrated bonding resin has a moisture content between about 5% and about 65% water by weight of total binder. In one embodiment, the solvent may be less than 50% of the total weight of the remaining material. In one embodiment, the solvent may be less than 35% of the total weight of the remaining material. In one embodiment, a substantially dehydrated bonding resin has between about 10% and about 35% water by weight of total bonding resin. In one embodiment, the solvent may comprise less than about 20% of the total weight of the remaining material.

As used herein, the term cured bonding resin describes the polymeric product of curing the uncured bonding resin. The cured bonding resin may have a characteristic brown to black color. While described as brown or black, another characteristic is that the binder tends to absorb light over a broad range of wavelengths. As the polymer of the cured bonding resin is extensively cross-linked, the cured bonding resin is substantially insoluble. For example, the bonding resin is predominantly insoluble in water. As described herein, the uncured bonding resin provides sufficient binding capacity to consolidate fibers; however, the cured bonding resin imparts the robust, long-lasting durability and physical properties commonly associated with cross-linked polymers.

The bonding resin reactants described herein are soluble in water and when combined in water, a binder solution is obtained. In one embodiment, a surfactant is included in the aqueous solution to increase the solubility or dispersability of one or more bonding resin reactants or additives. For example, a surfactant may be added to the aqueous binder solution to enhance the dispersibility of a particulate additive. In one embodiment, a surfactant is used to create an emulsion with a non-polar additive or binder reactant. In one embodiment, the binder solution comprises about 0.01% to about 5% surfactant by weight based on the weight of the binder solution.

Preferably, the components for the bonding resin are mixed less than 1 hour before being used and cured to become a bonding resin. A particular advantage of the present invention is that no pre-reaction between lignin and/or tannin and hydroxymethylfurfural, furfural, furfuryl alcohol, acetoxymethyl furfural or an oligomer of hydroxymethylfurfural is needed. More specifically, the present invention does not require any pre-reaction at elevated temperature, such as a pre-reaction at a temperature of 40-170° C. According to the present invention, the hydroxymethylfurfural (HMF), furfural (FU), furfuryl alcohol (FA) or acetoxymethyl furfural reacts with polyamine and lignin and/or tannin.

The binder solutions described herein can be applied to fibrous material (e.g., sprayed onto a mat or sprayed onto the fibers as they enter the forming region), during production of fibrous insulation products. Once the binder solution is in contact with the mineral fibers the residual heat from the mineral fibers (note that glass fibers for example are made from molten glass and thus contain residual heat) and the flow of air through and/or around the product will cause a portion of the water to evaporate from the binder solution. Removing the water leaves the remaining components of the bonding resin on the fibers as a coating of viscous or semi-viscous high-solids mixture. This coating of viscous or semi-viscous high-solids mixture functions as a bonding resin. At this point, the mat has not been cured. In other words, the uncured bonding resin functions to bind the fibers in the mat.

The above described uncured bonding resins can be cured. For example, the process of manufacturing a cured insulation product may include a subsequent step in which heat is applied as to cause a chemical reaction in the uncured bonding resin. For example, in the case of making fiberglass insulation products or other mineral fiber insulating products, after the binder solution has been applied to the fibers and dehydrated, the uncured insulation product may be transferred to a curing oven. In the curing oven the uncured insulation product is heated, typically from about 60° C. to about 320° C., such as from 60° C. to 250° C. or from 100° C. to 250° C., causing the bonding resin to cure. The cured bonding resin is thus a formaldehyde-free, water-resistant bonding resin that binds the fibers of the fibrous insulation product together. The drying and thermal curing may occur either sequentially, simultaneously, contemporaneously, or concurrently.

An uncured fiber product typically comprises about 3% to about 40% of dry binder solids (total uncured solids by weight). In one embodiment, the uncured fiber product comprises about 5% to about 25% of dry binder solids. In one embodiment, the uncured fiber product comprises about 50% to about 97% fibers by weight.

A cured bonding resin is the product of curing the bonding resin. The term cured indicates that the bonding resin has been exposed to conditions that initiate a chemical change. Examples of these chemical changes may include, but are not limited to, (i) covalent bonding, (ii) hydrogen bonding of binder components, and (iii) chemically cross-linking the polymers and/or oligomers in the bonding resin. These changes may increase the bonding resin's durability and solvent resistance as compared to the uncured bonding resin. Curing a bonding resin may result in the formation of a thermoset material. In addition, a cured bonding resin may result in an increase in adhesion between the matter in a collection as compared to an uncured bonding resin. Curing can be initiated by, for example, heat, microwave radiation, and/or conditions that initiate one or more of the chemical changes mentioned above.

In a situation where the chemical change in the bonding resin results in the release of water, e.g., polymerization and cross-linking, a cure can be determined by the amount of water released above that which would occur from drying alone. The techniques used to measure the amount of water released during drying as compared to when a bonding resin is cured, are well known in the art.

The bonding can also be used in the manufacture of wood fiber insulation, laminates and wood products such as plywood, oriented strandboard (OSB), laminated veneer lumber (LVL), medium density fiberboards (MDF), high density fiberboards (HDF), parquet flooring, curved plywood, veneered particleboards, veneered MDF or particle boards. The present invention is also directed to such wood fiber insulation, laminates, wood products such as plywood, oriented strandboard (OSB), laminated veneer lumber (LVL), medium density fiberboards (MDF), high density fiberboards (HDF), parquet flooring, curved plywood, veneered particleboards, veneered MDF or particle boards manufactured using the bonding resin. The bonding resin according to the present invention may also be used in the manufacture of composites, molding compounds and foundry applications.

### EXAMPLES

### Example 1

Lignin solution was prepared first by adding 211 g of powder lignin (solid content 95%) and 685 g of water to a 1 L glass reactor at ambient temperature and stirred until the lignin was fully and evenly dispersed. Then, 104 g of 28-30% ammonia solution was added to the lignin dispersion. The composition was stirred for 60 minutes to make sure that the lignin was completely dissolved.

### Example 2

3-Aminopropyl trimethoxysilane was diluted to 1% solution in water. Binder composition was prepared by weighing 60 g of lignin-ammonia solution from the example 1, 4.8 g of Hydroxymethyl furfural, 1.2 g of Jeffamine T403 (polyether amine) and 4 g of 1% of 3-aminopropyl trimethoxysilane into a 250 ml plastic container and was stirred with a wooden stick for 2 minutes. Then, 450 g glass beads was weighed into a beaker and the lignin mixture were poured on top of the glass beads and mixed for 2 minutes. Then, the glass beads bars were prepared by putting the glass beads-binder mixture into a silicon mould for baking in an oven at 200° C. for 1 hours. All glass beads bars were hard and stable after curing in the oven. The size of the bar for each test is height×thickness×length: 26 mm×18 mm×103 mm.

glass beads bars were post-cured for 24 hours and soaked in a water bath at 80° C. for 2 hours.

The glass beads bars were evaluated with 3-point bending test. The flexural strength before and after water soaking is given in the Table 1.

TABLE 1
Flexural Strength of the glass beads
bars with and without conditioning

| | Flexural Strength | Flexural Strength |
|---|---|---|
| | without conditioning | after conditioning |
| | [MPa] | [MPa] |
| Glass beads bars | 5.5 | 4.4 |

### Example 3

3-Aminopropyl trimethoxysilane was diluted to 1% solution in water. Binder composition was prepared by weighing 45 g of lignin-ammonia solution from the example 1, 7.2 g of Hydroxymethyl furfural, 1.8 g of Jeffamine T403 (polyether amine) and 4 g of 1% of 3-aminopropyl trimethoxysilane into a 250 ml plastic container and was stirred with a wooden stick for 2 minutes. Then, 450 g glass beads was weighed into a beaker and the lignin mixture were poured on top of the glass beads and mixed for 2 minutes. Then, the glass beads bars were prepared by putting the glass beads-binder mixture into a silicon mould for baking in an oven at 200° C. for 1 hours. All glass beads bars were hard and stable after curing in the oven. The size of the bar for each test is height×thickness×length: 26 mm×18 mm×103 mm.

Glass beads bars were post-cured for 24 hours and soaked in a water bath at 80° C. for 2 hours.

The glass beads bars were evaluated with 3-point bending test. The flexural strength before and after water soaking is given in the Table 2.

TABLE 2
Flexural Strength of the glass beads
bars with and without conditioning

| | Flexural Strength | Flexural Strength |
|---|---|---|
| | without conditioning | after conditioning |
| | [MPa] | [MPa] |
| Glass beads bars | 6.2 | 7.1 |

### Example 4

3-Aminopropyl trimethoxysilane was diluted to 1% solution in water. Binder composition was prepared by weighing 69 g of lignin-ammonia solution from the example 1, 3.5 g of Hydroxymethyl furfural, 0.7 g of Jeffamine T403 (polyether amine) and 4 g of 1% of 3-aminopropyl trimethoxysilane into a 250 ml plastic container and was stirred with a wooden stick for 2 minutes. Then, 450 g glass beads was weighed into a beaker and the lignin mixture were poured on top of the glass beads and mixed for 2 minutes. Then, the glass beads bars were prepared by putting the glass beads-binder mixture into a silicon mould for baking in an oven at 200° C. for 1 hours. All glass beads bars were hard and stable after curing in the oven. The size of the bar for each test is height×thickness×length: 26 mm×18 mm×103 mm.

Glass beads bars were post-cured for 24 hours and soaked in a water bath at 80° C. for 2 hours.

The glass beads bars were evaluated with 3-point bending test. The flexural strength before and after water soaking is given in the Table 3.

TABLE 3
Flexural Strength of the glass beads
bars with and without conditioning

| | Flexural Strength | Flexural Strength |
|---|---|---|
| | without conditioning | after conditioning |
| | [MPa] | [MPa] |
| Glass beads bars | 3.6 | 2.4 |

### Example 4

3-Aminopropyl trimethoxysilane was diluted to 1% solution in water. Binder composition was prepared by weighing 60 g of lignin-ammonia solution from the example 1, 3 g of Hydroxymethyl furfural, 3 g of Jeffamine T403 (polyether amine) and 4 g of 1% of 3-aminopropyl trimethoxysilane into a 250 ml plastic container and was stirred with a wooden stick for 2 minutes. Then, 450 g glass beads was weighed into a beaker and the lignin mixture were poured on top of the glass beads and mixed for 2 minutes. Then, the glass beads bars were prepared by putting the glass beads-binder mixture into a silicon mould for baking in an oven at 200° C. for 1 hours. All glass beads bars were hard and stable after curing in the oven. The size of the bar for each test is height×thickness×length: 26 mm×18 mm×103 mm.

Glass beads bars were post-cured for 24 hours and soaked in a water bath at 80° C. for 2 hours.

The glass beads bars were evaluated with 3-point bending test. The flexural strength before and after water soaking is given in the Table 4.

TABLE 4
Flexural Strength of the glass beads
bars with and without conditioning

| | Flexural Strength | Flexural Strength |
|---|---|---|
| | without conditioning | after conditioning |
| | [MPa] | [MPa] |
| Glass beads bars | 6.1 | 4.3 |

In view of the above detailed description of the present invention, other modifications and variations will become apparent to those skilled in the art. However, it should be apparent that such other modifications and variations may be effected without departing from the spirit and scope of the invention.

1. A bonding resin comprising:
a reaction product of
hydroxymethylfurfural, furfural, furfuryl alcohol, acetoxymethyl furfural or an oligomer of hydroxymethylfurfural or a combination thereof,
lignin, tannin, or both, and
a polyamine,
wherein the polyamine is a primary polyamine selected from a group consisting of: a diamine, triamine, tetraamine and pentaamine, and
wherein the polyamine is H₂N-Q-NH₂,
wherein Q is C₁-C₁₀ alkyl, cycloalkyl, C₁-C₁₀ heteroalkyl, or cycloheteroalkyl, each of which is optionally substituted,
wherein the lignin, the tannin or both are provided as a solution,
wherein a total amount of the lignin and the tannin in the bonding resin, calculated on a basis of dry lignin and dry tannin and dry bonding resin, is in a range of from 1 wt-% to 80 wt-%.

2. The bonding resin according to claim 1, further comprising:
a coupling agent.

3. The bonding resin according to claim 1, wherein the polyamine is selected from a group consisting of: 1,6-diaminohexane, 1,5-diamino-2-methylpentane, hexamethylenediamine, polyetheramine, 3-(Aminomethyl)-3,5,5-trimethylcyclohexan-1-amine, diethylenetriamine, 1-piperazineethaneamine, bis(hexamethylene)triamine, triethylenetetramine, and tetraethylenepentamine.

4. The bonding resin according to claim 1, wherein the polyamine is a polyether amine.

5. A The bonding resin according to claim 1, wherein the bonding resin does not comprise any epoxy-based crosslinker.

6. A The bonding resin according to claim 1, wherein the lignin is not chemically modified.

7. The bonding resin according to claim 1, wherein a weight ratio of the lignin and the tannin to the polyamine is in a range of from 1:50 to 50:1, calculated on a basis of dry solids.

8. The bonding resin according to claim 1, wherein an amount of hydroxymethylfurfural (HMF), furfural (FU), furfuryl alcohol (FA), acetoxymethyl furfural or oligomer of HMF in the bonding resin is between 10-80 wt-%, calculated as a total dry weight of hydroxymethylfurfural (HMF), furfural (FU), furfuryl alcohol (FA), acetoxymethyl furfural or oligomer of HMF and a total weight of the bonding resin.

9. The bonding resin according to claim 1, wherein an amount of lignin in the bonding resin, calculated on a basis of dry lignin and dry bonding resin, is in a range of from 1 wt-% to 30 wt-%.

10. A fibrous insulation product comprising:
the bonding resin according to claim 1, and
a fibrous material.

11. The fibrous insulation product according to claim 10, wherein the fibrous material is selected from a group consisting of: wood fibers glass fibers, mineral fibers, aramid fibers, ceramic fibers, metal fibers, carbon fibers, polyimide fibers, polyester fibers, rayon fibers, and cellulose fibers.

12. A wood fiber insulation, laminate, wood product manufactured using the bonding resin according to claim 1.

13. A process for preparing a bonding resin, comprising the steps of:
mixing hydroxymethylfurfural, furfural, furfuryl alcohol, acetoxymethyl furfural or an oligomer of hydroxymethylfurfural or a combination thereof, with lignin, or tannin, or both, and with a polyamine,
wherein the polyamine is a primary polyamine selected from a group consisting of: a diamine, triamine, tetraamine and pentaamine,
wherein the polyamine is H₂N-Q-NH₂,
wherein Q is C₁-C₁₀ alkyl, cycloalkyl, C₁-C₁₀ heteroalkyl, or cycloheteroalkyl, each of which is optionally substituted,
wherein the lignin, the tannin, or both are provided as a solution, and
wherein a total amount of lignin in the bonding resin, calculated on a basis of dry lignin and dry tannin and dry bonding resin, is in a range of from 1 wt-% to 50 wt-%, and,
wherein components for the bonding resin are mixed and combined less than 1 hour before being cured to form the bonding resin.
